(19) United States
(12) Patent Application Publication
(10) Pub. No.: US 2023/0048869 A1
(43) Pub. Date: Feb. 16, 2023

(54) FREEZE-DRIED PREPARATION OF CHEMILUMINESCENT IMMUNE MICROSPHERES, AND PREPARATION METHOD AND APPLICATION THEREOF

(71) Applicant: Anbio (Xiamen) Biotechnology Co., Ltd, Fujian (CN)

(72) Inventors: Zhujin Chen, Fujian (CN); Tao Cheng, Fujian (CN); Jieli Zhang, Fujian (CN); Linhua Zhang, Fujian (CN); Xiao Hu, Fujian (CN); Yang Lei, Fujian (CN)

(73) Assignee: Anbio (Xiamen) Biotechnology Co., Ltd, Fujian (CN)

(21) Appl. No.: 17/965,761

(22) Filed: Oct. 13, 2022

Related U.S. Application Data
(63) Continuation of application No. PCT/CN2021/110438, filed on Aug. 4, 2021.

(30) Foreign Application Priority Data
Dec. 18, 2020 (CN) 202011500680.5
Dec. 17, 2021 (CN) 202111553600.7

(51) Int. Cl. G01N 33/532; G01N 33/543; G01N 33/68; G01N 33/74

(52) U.S. Cl. CPC G01N 33/532; G01N 33/54326; G01N 33/6887; G01N 33/74

(57) ABSTRACT

The present invention discloses a freeze-dried preparation of chemiluminescent immune microspheres, and a preparation method and an application thereof. The preparation is composed of one or more spherical solid particles having the same composition, which are uniform, smooth, and highly stable. In the preparation process, a magnetic particle coating raw material, an acridinium ester marking raw material and a reagent storage agent are freeze-dried into microspheres, so that the freeze-dried preparation can be stored and transported at normal temperature; the microspheres prepared in the present invention can be sub-packaged conveniently into individual packs, which are hygienic, simple and clear, and convenient to get and use, thereby the safety and convenience of use are improved. The preparation provided by the present invention and the test kit prepared according to the present invention can be used for immunoassays that are not for a disease diagnoses or treatment purpose.

### CROSS-REFERENCE TO RELATED APPLICATIONS

This application is a continuation of international PCT application serial no. PCT/CN2021/110438, filed on Aug. 4, 2021, which claims the priority benefit of China application serial no. 202011500680.5, filed on Dec. 18, 2020. This application also claims the priority benefit of China application serial no. 202111553600.7, filed on Dec. 17, 2021. The entirety of each of the above-mentioned patent applications is hereby incorporated by reference herein and made a part of this specification.

### BACKGROUND OF THE INVENTION

### 1. Technical Field

The present invention relates to the technical field of immunoassay, in particular to a freeze-dried preparation of chemiluminescent immune microspheres, and a preparation method and an application thereof.

### 2. Background Art

Chemiluminescent immunoassay (CLIA), also known as chemiluminescent labeling immunoassay, is to use a chemiluminescent agent to directly label an antigen or an antibody (chemiluminescent marker), and react with the corresponding antibody or antigen or magnetic particle antigen or antibody in the specimen to be tested, separate the chemiluminescent marker in a bound state (a precipitated part) from the chemiluminescent marker in a free state under the action of a magnetic field, then add a luminescence accelerator to have a luminescence-producing reaction, and detect the target substance by means of detecting the luminescence intensity.

However, there are rigorous requirements for the conditions of storage and transportation of the reagents for chemiluminescent immunoassay. Usually, luminescent immunoreagents must be stored at 2-8° C., and transported through a cold chain. If those conditions are not met, the performance of the luminescent immunoreagents will be severely affected, and even the reagents may fail.

### SUMMARY OF THE INVENTION

An object of the present invention is to provide a freeze-dried preparation of chemiluminescent immune microspheres, and a preparation method and an application thereof.

The technical problem to be solved by the present invention is that the reagents for luminescent immunoassay can't be stored and transported at normal temperature.

To attain the object described above, the present invention employs the following technical schemes:

A freeze-dried preparation of chemiluminescent immune microspheres composed of one or more spherical solid particles having the same composition, comprising a reagent storage agent, a magnetic particle coating raw material, and an acridinium ester marking raw material, wherein the magnetic particle coating raw material is an antibody or an antigen coupled with a magnetic particle, and the acridinium ester marking raw material is an antibody or an antigen marked by an acridinium ester.

Furthermore, the reagent storage agent comprises a freeze-drying protectant, PEG20000 and an antioxidant, wherein the freeze-drying protectant comprises mannitol, trehalose, casein, surfactant, gelatin, preservative, and buffer solution TBS.

The PEG20000 acts as a cryoprotectant and a dehydration protectant on the one hand, and can accelerate the reaction and improve the detection sensitivity of the preparation in the immune reaction on the other hand (to protect the stability of the preparation, PEG20000 is added to accelerate the immunologic reaction and improve the sensitivity of the reagent in view that the complicated components of the preparation storage agent prepared/used for the preparation tends to decrease the signal value of the preparation reaction); the antioxidants may be sodium thiosulfate and EDTA-2Na, which prevent the reagent from oxidative deterioration during freeze-drying and storage.

In the freeze-drying protectant, the mannitol is used as a freeze-drying filler, has no hygroscopicity, can achieve rapid freeze-drying, can prevent the active components from sublimating with water vapour, and can shape the active components; the trehalose plays the role of a cryoprotectant in the freezing process and the role of a dehydration protectant in the drying process; the casein protects the proteins, serves as a filler in the dehydration and drying process, and controls the background and eliminates false positive in the immunoreagent; the surfactant may be Tween 20, Tween 80 or Triton x100, which reduces the freezing and dehydration deformation resulted from the ice-water interface tension in the freeze-drying process, attains the effects of a wetting agent and a refolding agent for the active components in the rehydration process, and reduces non-specific reactions in the immunoreaction process at the same time; the gelatin serves as a freeze-drying filler, and can block the sites in the immunoreaction, improves the stability of the proteins, and eliminates non-specific reactions; and the preservative may be Proclin 300, which has broad-spectrum bacteriostasis and high biocompatibility.

Furthermore, based on the total mass percentage of the freeze-drying protectant, which is regarded as 100%, the mass percentages of the components in the freeze-drying protectant are as follows: mannitol: 2%-10%; trehalose: 5%-20%; casein: 0.5%-2%; surfactant: 0.05%-0.5%; gelatin: 0.05%-1%; preservative: 0.1%-1%; and buffer solution TBS: 65.5%-92.3%.

Furthermore, the buffer solution TBS is made of 10-50 mm Tris-HCl and 0.85% NaCl, so that the pH of the buffer solution is neutral, specifically pH=7.0-7.4; the antioxidant is a mixture of sodium thiosulfate and EDTA-2Na mixed at 1:1 mix ratio.

The present invention further provides a method for preparing the freeze-dried preparation of chemiluminescent immune microspheres, which comprises the following steps:

S1: obtaining a reagent storage agent, which comprises a freeze-drying protectant, PEG20000 and an antioxidant;

S2: obtaining a magnetic particle coating raw material and an acridinium ester marking raw material, wherein the magnetic particle coating raw material is an antibody or an antigen coupled with a magnetic particle, and the acridinium ester marking raw material is an antibody or an antigen marked by an acridinium ester; mixing the magnetic particle coating raw material with the reagent storage agent obtained in the step S1 to form a reagent of magnetic particle coating raw material, and mixing the acridinium ester marking raw material with the reagent storage agent obtained in the step S1 to form a reagent of acridinium ester marking raw material;

S3: mixing the reagent of magnetic particle coating raw material and the reagent of acridinium ester marking raw material that are obtained in the step S2 at a mix ratio of 1:1 to form a mixed solution, spotting the mixed solution on a liquid nitrogen spotter to form frozen microspheres, and then transferring the frozen microspheres into a freeze drier and freeze-drying the frozen microspheres in vacuum, so as to obtain the freeze-dried preparation of microspheres composed of spherical solid particles.

Furthermore, the method for preparing a freeze-dried preparation of chemiluminescent immune microspheres described above further comprises the following step:

S4: charging a protective gas to the freeze-dried preparation of microspheres composed of spherical solid particles that are obtained in the step S3, sub-packaging the freeze-dried preparation of microspheres into individual packs, and storing the individual packs for assay.

The present invention further provides an application of the aforesaid freeze-dried preparation of chemiluminescent immune microspheres in the preparation of an immunoassay kit.

The present invention further provides a chemiluminescent immunoassay test kit, which contains the aforesaid freeze-dried preparation of chemiluminescent immune microspheres.

Furthermore, the kit further comprises deionized water for disintegrating the freeze-dried preparation of chemiluminescent immune microspheres.

The present invention further provides an application of the aforesaid freeze-dried preparation of chemiluminescent immune microspheres or the aforesaid chemiluminescent immunoassay test kit for immunoassays that are not for a disease diagnoses or treatment purpose, and the test item may be the content of n-terminal pro-brain natriuretic peptide NT-proBNP, or the content of prostate specific antigen PSA, or the content of thyroid-stimulating hormone TSH, or the content of cardiac troponin-I cTnI, or the content of procalcitonin PCT in serum or plasma.

Compared with the prior art, the present invention attains the following beneficial effects:

1. The freeze-dried preparation of chemiluminescent immune microspheres provided by the present invention is composed of one or more spherical solid particles having the same composition, which are uniform and smooth, and have high stability. The freeze-dried preparation effectively solves the problem that chemiluminescent immunoreagents can't be stored and transported at normal temperature. To use the freeze-dried preparation, simply purified water has to be added to the freeze-dried preparation, thus the microspheres are quickly disintegrated and re-dissolved into a homogeneous suspension with magnetic bead powder. The magnetic beads (magnetic particles) do not agglomerate, and the liquid has no undissolved substance except the magnetic bead powder.

2. The freeze-dried preparation of chemiluminescent immune microspheres provided by the present invention contains a reagent storage agent composed of specific components in specific proportions, which protects the magnetic particle coating raw material and the acridinium ester marking raw material in terms of environmental factors including temperature, humidity and pH value, etc., avoids or mitigates various stress damages to the raw materials in the freeze-drying process, protects the reagent against denaturation and deactivation, and protects the stability of the reagent.

3. The preparation method of the freeze-dried preparation of chemiluminescent immune microspheres provided by the present invention is simply and convenient. With the preparation method, the magnetic particle coating raw material, the acridinium ester marking raw material and the reagent storage agent are freeze-dried to form a microsphere, which can be transported and stored at normal temperature. The magnetic particle in the magnetic particle coating raw material is a solid phase carrier, the acridinium ester marking raw material is a liquid phase, and the solid phase carrier and the liquid phase are freeze-dried on the same microsphere. In view that the freeze-dried preparation may be subjected to various stress damages in the freeze-drying process, the specific freeze-drying protectant in the present invention is added to avoid uneven surface pores of the freeze-dried microsphere resulted from the solid phase carrier, decrease the hygroscopicity of the chemiluminescence immunoreagent, avoid adverse effects to the structure of the preparation, thereby protect the stability of the preparation; the microspheres prepared in the present invention can be sub-packaged conveniently into individual packs, which are hygienic, simple and clear, and convenient to get and use, thereby the problems of pairing and confusion during mixed packaging and use are avoided; the interferences and influences of other substances during transportation and use are avoided, the safety and convenience are improved, and the preparation is more favored by the users and can be popularized easily in the market.

4. The freeze-dried preparation of chemiluminescent immune microspheres provided by the present invention can be used for immunoassays that are not for a disease diagnoses or treatment purpose, the test item may be the content of n-terminal pro-brain natriuretic peptide NT-proBNP, or the content of prostate specific antigen PSA, or the content of thyroid-stimulating hormone TSH, or the content of cardiac troponin-I cTnI, or the content of procalcitonin PCT in serum or plasma. The freeze-dried preparation provided by the present invention has high sensitivity, high specificity, and a wide linearity range, and can meet the demand for clinical assays.

### BRIEF DESCRIPTION OF THE DRAWINGS

FIG. 1 is a schematic diagram of the appearance of the preparation provided in an example 1 of the present invention;

FIG. 2 is a standard curve chart of human serum NT-proBNP determined with the preparation provided in the example 1 of the present invention; and

FIG. 3 is a diagram of correlation between the test result of the preparation provided in the example 1 of the present invention and the Roche electrochemical test result.

### DETAILED DESCRIPTION OF THE INVENTION

There is a technical problem that the reagents for luminescent immunoassay and analysis can't be stored and transported at normal temperature in the prior art.

In view of that problem, in the present invention, a novel technical scheme is proposed, with which a freeze-dried preparation of chemiluminescent immune microspheres that can be stored and transported at normal temperature, and a preparation method and an application thereof are provided.

To make the present invention understood more clearly, hereunder the present invention will be detailed in embodiments with reference to the accompanying drawings.

### Example 1

In this example, a freeze-dried preparation of chemiluminescent immune microspheres and a test kit that can be used to measure the content of N-terminal pro-brain natriuretic peptide (NT-proBNP) in human serum quantitatively, a preparation method and test method of the freeze-dried preparation of chemiluminescent immune microspheres, and a test result, are provided.

Brain Natriuretic Peptides (BNPs) actually mainly come from the ventricles, and gained the name because they were first extracted from swine brain by a Japanese scholar Sudoh in 1988. BNPs are mainly synthesized and secreted by ventricular myocytes, and have a strong vasodilatation effect. The ventricular myocyte secretion system is a major endocrine system for human body to resist volume overload and hypertension. An increased load on the ventricles can lead to BNPs release. N-terminal pro-brain natriuretic peptide (NT-proBNP) is one of the pro-brain natriuretic peptides. It is an inactive N-terminal fragment of BNP prohormone after division, and is mainly secreted in the left ventricle. Compared with BNPs, NT-proBNP has a longer half-life and is more stable, and the concentration of NT-proBNP can reflect the release of BNPs newly synthesized in a short time rather than stored BNPs. Researches have shown that the level of serum NT-proBNP is of great value in the diagnosis of hypertension, prognosis judgment and treatment guidance of heart failure.

NT-proBNP can be measured quantitatively with a chemiluminescent method. However, there are rigorous requirements for the conditions of storage and transportation of conventional chemiluminescent immunoreagents. Usually, chemiluminescent immunoreagents must be stored at 2-8° C. and transported through a cold chain. If those conditions are not met, the performance of the luminescent immunoreagents will be severely affected, and even the reagents may fail. The freeze-dried preparation of chemiluminescent immune microspheres provided in this example can be stored at room temperature and individually packaged. In this example, NT-proBNP Ab1 is coupled with magnetic microspheres modified by a carboxyl group, NT-proBNP Ab2 is labeled by acridine sulfonamide, and a reagent storage agent is added to NT-proBNP Ab1 and NT-proBNP Ab2 respectively. The two substances are mixed and spotted on a spotter with liquid nitrogen to form frozen microspheres, then the frozen microspheres are transferred into a freeze drier and freeze-dried in vacuum; then a protective gas is charged to the freeze-dried microspheres, and the freeze-dried microspheres are sub-packaged and stored. During the test, purified water is added to the sample to be tested, the NT-proBNP antigen in the sample to be tested is mixed with the preparation, a double-antibody sandwich complex is formed and bonded on the magnetic bead, and then washed and separated. The complex is luminescent under the action of an excitation liquid, and the relative luminescence intensity (RLU) is positive correlated with the concentration of the NT-proBNP antigen in the sample; thus, individually packaged preparation of freeze-dried microspheres for quantitative measurement of N-terminal pro-brain natriuretic peptide (NT-proBNP) by direct chemiluminescent immunoassay is developed.

The names and sources of the reagents and materials used in this example are as follows: NT-proBNP antibody pairing (Fapon Bio-Co., Ltd.), NT-proBNP calibrator (Shanghai Linc-Bio Science Co., Ltd.), human serum albumin, bilirubin, protoheme and triglyceride (Sigma-Aldrich); magnetic microspheres (carboxyl group, 2.9 μm, JSR Co., Ltd., Japan), acridine sulfonamide (NSP-SA-NHS Shenzhen Maxchemtech Co., Ltd.), desalting column (Thermo fisher), dimethylformamide DMSO (Sigma-Aldrich), lysine (Sigma-Aldrich), casein, bovine serum albumin BSA (Sigma-Aldrich), mannitol, trehalose, gelatin, sodium thiosulfate and disodium ethylenediamine tetraacetate (Shanghai Aladdin Bio-Chem Technology Co., Ltd.) and PEG20000 (Sinopharm Chemical Reagent Co., Ltd.).

The instruments and devices used in this example include: automatic chemiluminescence analyzer (KEYSMILE SMART 500), high performance liquid chromatograph (Agilent Technologies 1260 Infinity II), Berthold Chemiluminescence Analyzer (Centro LB 960), and TECAN magnetic particle washing machine (Hydro FLEX).

The test sample in this example is as follows: 100 parts of calibrated human serum (from Fujian Medical University Union Hospital, the calibration result was measured with a Roche electrochemiluminescence method).

The implementation process of this example is as follows:

1. Solution preparation

(1) Preparation of reagent storage agent: 5% mannitol, 10% trehalose, 1% casein, 0.05% Tween 20, 0.5% gelatin and 0.1% preservative were dissolved and diluted with 83.3% 25 mm TBS buffer with pH=7.0-7.4 to respective concentrations. Then 5 mmol/mL sodium thiosulfate, 5 mmol/mL disodium ethylenediamine tetraacetate, and 0.1% PEG20000 were added, and the above components are mixed homogeneously.

(2) Preparation of excitation liquids: excitation liquid A: 0.1% H₂O₂+0.1 mol/L HNO₃; excitation liquid B: 0.2 mol/L NaOH+1% Triton X-100.

2. Reagent 1: preparation of NT-proBNP-Ab1 coupled with magnetic microsphere

10 mg carboxyl modified magnetic microsphere suspension was loaded in a sample tube, 900 μL 0.1 mol/mL MES solution with pH=5.0 was added into the sample tube, and the mixture was mixed homogenously; then 100 μL 10 mg/mL EDC solution and 200 μL 10 mg/mL NHS solution were added respectively, and the mixture was held at room temperature for 30 minutes for reaction to activate the magnetic microspheres; the supernatant was removed, and the magnetic beads were resuspended in 1 mL 0.1 mol/mL MES solution with pH=5.0; then 100 μg SCC-ab1 was directly added and the solution was incubated at 37° C. for 3 hours; the supernatant was removed, 50 μL 10 mg/mL BSA was added and the mixture was sealed at room temperature for 1 hour, then the mixture was washed with 0.025 mol/mL TBST buffer for 4 times to remove the free antibody, 10 mL reagent storage agent was added to prepare a reagent I, which was the aforesaid reagent of magnetic particle coating raw material.

3. Reagent 2: preparation of NT-proBNP-Ab2 labeled with acridine sulfonamide

An appropriate amount of antibody was obtained and diluted to 1 mg/mL with 50 mm CB with pH=9.6, and purified in a desalting column; the mole number of the antibody was calculated, NSP-SA-NHS (diluted with DMSO) in quantity of 15 times the mole number was added into the purified antibody, and the mixture was kept in a dark environment at 4° C. for 1 hour for reaction; lysine in quantity of 20 times the mole number of the antibody was added, and the mixture was kept in a dark environment for 30 minutes for reaction; the labeled complex was purified with a high performance liquid chromatograph (mobile phase: 0.1 mol/L PBS buffer with pH=6.5, chromatographic column: molecular sieve chromatographic column, UV: 280 nm), glycerol in the same volume was added, and the mixture was frozen and stored at −20° C.; before use, the mixture was diluted with a reagent storage agent to 0.001 mg/mL antibody concentration and prepared into a reagent II, which was the aforesaid reagent of acridinium ester marking raw material.

4. Spotting and freeze-drying of the reagent

The reagent I and the reagent II were mixed at 1:1 mix ratio, the resulting mixture was added into a liquid nitrogen spotter, the spotting amount was set to 20 μL per drop, spotting was carried out to form frozen microspheres, the frozen microspheres were transferred into a freeze drier and freeze-dried to obtain a freeze-dried preparation of microspheres, then a protective gas was charged, and the freeze-dried preparation of microspheres was sub-packaged and stored.

5. Testing and analysis

30 μL sample to be tested and 120 μL deionized water were added into the sample cup containing the freeze-dried preparation of microspheres obtained in step 4; the mixture was incubated at 37° C. for 10 minutes, and then was washed for 3 times with a washing liquid; then 100 μL excitation liquid A and 100 μL excitation liquid B were added, and the relative luminescence intensity was measured. The parameters of the automatic chemiluminescence analyzer were set according to the above steps.

In the immunoreaction, the enterprise reference was measured in three wells in parallel, and the mean value was used; the relative luminescence intensity (RLU) and corresponding concentration were fitted linearly, and a master calibration curve was established and scanned into the analyzer. The test result of the patient was determined automatically by the system software during the test of the sample with reference to the stored calibration data. The result was presented in the form of pmol/mL.

6. Result of experiment

(1) Appearance and physical and chemical properties of the preparation

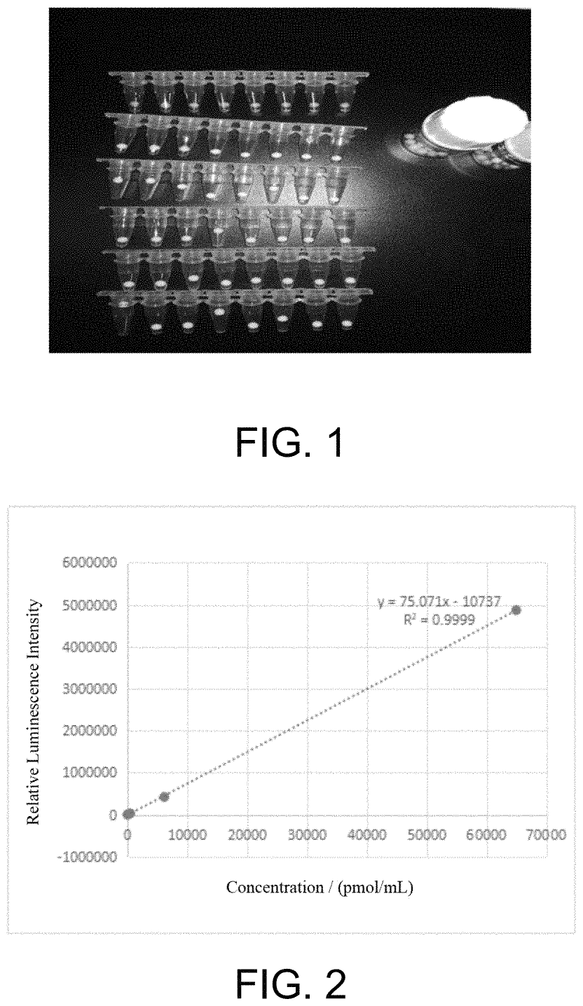

As shown in FIG. 1, the preparation is composed of uniform and smooth solid microspheres. When purified water is added to the preparation, the microspheres were disintegrated and re-dissolved quickly and formed a uniform suspension with magnetic bead powder. The magnetic beads didn't agglomerate, and the liquid had no undissolved substance except the magnetic bead powder.

(2) Linearity range

Standard samples (35,000 pmol/mL) close to the upper limit of the linearity range were diluted with normal human serum to 6 different concentrations, among which the minimum concentration was 20 pmol/mL, which was close to the lower limit of the linearity range. Each sample was tested for 3 times, the mean value was calculated, and the relative luminescence intensity was fitted linearly. As shown in FIG. 2, in the linearity range (20-35,000 pmol/mL), the linearly correlation coefficient R was greater than 0.999, which exhibited close correlation.

(3) Minimum detection limit

A calibrator at zero concentration was used as the sample and tested repeatedly for 20 times, the RLU values of the 20 detection results were obtained, and the mean value x̅ and standard deviation (SD) of the RLU values were calculated, the RLU value corresponding to x̅+2SD was put into the master calibration curve to calculate the corresponding concentration value, which should not be higher than 20 pmol/mL, and 20 pmol/mL was set to the minimum detection limit.

(4) Precision

Intra-batch precision: 1,000 pmol/mL enterprise reference and 10,000 pmol/mL enterprise reference were used, and the enterprise reference at each concentration was tested for 10 times in parallel, and the test result is shown in Table 1; the mean value x̅ and the standard deviation SD of the test result were calculated, and CV=x̅/SD*100%; then intra-batch coefficient of variation (CV) was smaller than 5%. CVs obtained: 4.8% at 1,000 pmol/ml; 3.2% at 10,000 pmol/mL.

TABLE 1
Test Result of Intra-Batch Precision.

| Number of test times | 1 | 2 | 3 | 4 | 5 |
|---|---|---|---|---|---|
| 1,000 pmol/mL | 73626 | 72913 | 74183 | 72095 | 73037 |
| 10,000 pmol/mL | 630022 | 667873 | 686925 | 664219 | 681297 |
| Number of test times | 6 | 7 | 8 | 9 | 10 |
| 1,000 pmol/mL | 65973 | 74857.5 | 76473 | 74227.5 | 66450 |
| 10,000 pmol/mL | 638059 | 692938 | 667815 | 659324 | 646513 |

(5) Recovery Ratio

Three high-value samples were added to three parts of normal human serum samples (all the endogenous test concentrations were lower than 100 pm/mL), and the volume ratio of the added high-value sample to the normal human serum was 1:9. The average recovery ratio was 95.7%. The test result of recovery ratio is shown in Table 2.

TABLE 2
Test Result of Recovery Ratio

| | Test result of normal | High-value | Actual value | Recovery | |
|---|---|---|---|---|---|
| Sample | human sample | sample | (after | ratio | Aver- |
| ID | (pmol/mL) | (pmol/mL) | conversion) | (%) | age |
| 1 | 95 | 13314 | 12219.5804 | 91.8 | 95.7 |
| 2 | 48 | 15294 | 15840.52042 | 103.6 | |
| 3 | 58 | 7035 | 6452.915885 | 91.7 | |

(6) Stability

The freeze-dried preparation of chemiluminescent immune microspheres prepared in this example was stored at 45° C. for 90 days, the enterprise reference was tested, the relative luminescence intensity obtained in the test was compared with that before the storage, and the change ratio of thermostability after storage at 45° C. for 90 days was determined to be smaller than or equal to 10%; the preparation was stored under the specified preparation storage conditions (25±5° C.) for 5 months, the standard samples were tested, and the relative luminescence intensity was compared with that before the storage; the change ratio of thermostability was determined to be smaller than or equal to 10% (see Table 3 for the test result). Compared with conventional chemiluminescent reagents, the stability was remarkably improved. The change ratio of thermostability of conventional chemiluminescent reagents (liquid phase) after storage at 45° C. for 3 days is about 20% (see Table 4 for the test result).

TABLE 3
Test Result of the Stability of the Preparation Provided in Example 1

| | Freeze-dried preparation of microspheres in the same batch | | | | |
|---|---|---|---|---|---|
| | | Test result after storage at | | Test result after storage at 25 ± | |
| | | 45° C. for 90 days | | 5° C. for 5 months | |
| | Test | | Change ratio | | Change ratio |
| | result at | | compared | | compared |
| | the time | | with the | | with the |
| | of | | value at | | value at the |
| | preparation | | the time of | | time of |
| | Tested | Tested | preparation | Tested | preparation |
| | RLU | RLU | (%) | RLU | (%) |
| Enterprise | 4841 | 4440 | −8.3 | 4747 | −1.9 |
| reference 1 | | | | | |
| Enterprise | 12231 | 11309 | −7.5 | 11079 | −9.4 |
| reference 2 | | | | | |
| Enterprise | 20841 | 19605 | −5.9 | 19765 | −5.2 |
| reference 3 | | | | | |

TABLE 4
Test Result of the Stability of Conventional Chemiluminescent Reagents
Conventional chemiluminescent reagents (liquid phase)

| | Test result at | Test result | Change ratio |
|---|---|---|---|
| | the time of | after storage at | compared with the |
| | preparation | 45° C. for 3 days | value at the time of |
| | Tested RLU | Tested RLU | preparation (%) |
| Enterprise reference 1 | 4756 | 4068 | −14.5 |
| Enterprise reference 2 | 12596 | 9671 | −23.2 |
| Enterprise reference 3 | 21855 | 16831 | −23.0 |

(7) Comparative test

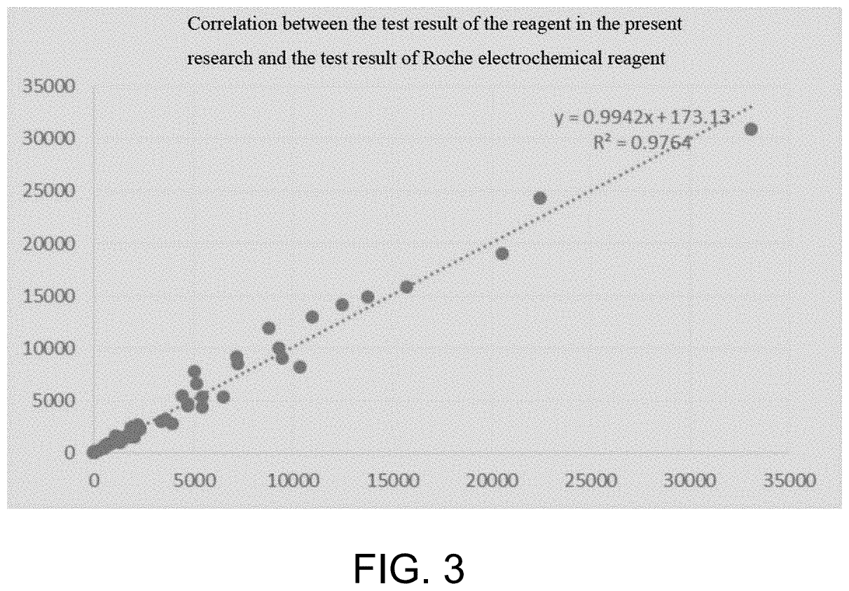

57 clinical samples from Fujian University Union Medical Hospital were collected and measured with a research kit, and the result was compared with the Roche electrochemiluminescence measurement result, as shown in FIG. 3. The regression equation was y=0.9942x+173.13, and the correlation coefficient was R²=0.9764, which indicated that there was close correlation between the test results of the content of the NT-proBNP antigen in the tested serum samples.

(8) Specificity

For the specificity of the preparation for testing, the influences of common interfering factors including human serum albumin, triglyceride, protoheme, bilirubin, and BNP on the test result were studied, all the measured values of the interfering substances were smaller than 20 pmol/mL (see Table 5), which indicates that the preparation has high specificity for testing.

TABLE 5
Test Result of the Specificity of the Preparation in Example 1

| Cross-reactive | Concentration of cross-reactive | | Measured value of cross reaction | |
|---|---|---|---|---|
| substance | substance | | substance | |
| Human serum albumin | 12 | g/dL | 9.5 | pmol/mL |
| Triglyceride | 3,000 | mg/dL | 0.5 | pmol/mL |
| Protoheme | 500 | mg/dL | 15 | pmol/mL |
| Bilirubin | 20 | mg/dL | 5 | pmol/mL |
| BNP | 3,000 | ng/mL | 19.5 | pmol/mL |

The freeze-dried preparation of chemiluminescent immune microspheres prepared in this example can be packed individually and stored at room temperature, and has the characteristics of high sensitivity, high specificity, wide linearity range, and high stability, etc., and can meet the need of clinical testing. An NT-proBNP test kit prepared from the freeze-dried preparation and purified water (deionized water) or excitation liquid can be used to detect the level of NT-proBNP, assist the clinical diagnosis and treatment of patients with heart failure, and provide reliable data support for the treatment of the patients.

### Example 2

Examples 2-5 provide the implementations of the freeze-dried preparation of chemiluminescent immune microspheres provided by the present invention for quantitative measurement of other immunoassay indicators. The immunoassay and analysis utilize a double-antibody sandwiching principle and employ a direct chemiluminescent immunoassay method as follows:

Incubation: the antigen in the sample, the antibody coupled with a magnetic microsphere, and the antibody labeled by acridine are bonded to form a double-antibody sandwich immune complex.

Washing: the unbound substances are separated from the immune complex by washing under the action of an applied magnetic field.

Excitation and reading: the immune complex is luminescent under the action of a pre-excitation liquid and an excitation liquid, and the chemiluminescent reaction is measured in relative light units.

The luminescent signal value of the sample to be tested is calculated with a calibration curve and the concentration of the antigen to be tested is obtained.

The preparation and testing processes are as follows:

With a direct chemiluminescent immunoassay method, a magnetic particle coating raw material (the magnetic particles are nanometer magnetic particles in size of 100-300 nm) and an acridinium ester marking raw material in which a reagent storage agent was added in advance were mixed (the coating method and the labeling method are conventional method in the art, and the coated or labeled antibody is the antibody corresponding to the target antigen to be detected), spotted on a liquid nitrogen spotter to form frozen microspheres, then the frozen microspheres were transferred into a freeze drier and freeze-dried in vacuum to obtain a freeze-dried preparation of microspheres composed of spherical solid particles, a protective gas is charged to the freeze-dried preparation of microspheres, and then the freeze-dried preparation of microspheres was sub-packaged and stored. During the test, a redissolving solution and the sample to be tested were added, and the antigen and the preparation were mixed to form a double-antibody sandwich complex bonded on the magnetic bead; then washing and separation were carried out. The experimental result indicated that the complex is luminescent under the action of the excitation liquid, and the relative luminescence intensity (RLU) is positively correlated with the concentration of the antigen in the sample.

The articles or test kit for the test may be those shown in Table 6. Alternatively, the test kit may only contain the freeze-dried preparation of chemiluminescent immune microspheres and/or deionized water (for Disintegration and redissolution of the preparation), while other articles are prepared additionally.

TABLE 6
Examples 2-5 Articles for Chemiluminescent Immunoassay and Analysis

| Name of reagent component | Raw materials |
|---|---|
| Freeze-dried microspheres | Contains antibody coupled with magnetic microspheres or antigen raw |
| | material 1; antibody labeled by acridinium ester or antigen raw material 2; |
| | and reagent storage agent |
| Quality control | Phosphate buffers containing antigen at different concentrations |
| Redissolution solution | Deionized water |
| Excitation liquid, | Excitation liquid A: 0.1% H₂O₂ + |
| | 0.1 mol/L HNO₃ |
| pre-excitation liquid | Excitation solution B: 0.2 mol/L |
| | NaOH + 1% Triton X-100 |
| Concentrated washing liquid | 0.2M PBST buffer |

In the Table 6, the reagent storage agent comprises a freeze-drying protectant, PEG20000, and an antioxidant, wherein the freeze-drying protectant comprises mannitol, trehalose, casein, surfactant, gelatin, preservative, and buffer solution TBS; based on the total mass percentage of the freeze-drying protectant, which is regarded as 100%, the mass percentages of the components are as follows: mannitol: 2%-10%; trehalose: 5%-20%; casein: 0.5%-2%; surfactant: 0.05%-0.5%; gelatin: 0.05%-1%; preservative: 0.1%-1%; and buffer solution TBS: 65.5%-92.3%. The specific mix ratios and addition amounts of the components of the reagent storage agent may be determined with reference to the example 1.

The example 2 provides a freeze-dried preparation of chemiluminescent immune microspheres and a test kit for testing and analyzing the total prostate (t-PSA) specific antigen in serum and/or plasma. The in vitro quantitative measurement of t-PSA-specific antigen in human serum or plasma may be used in combination with digital rectal examination (DRE) to assist the examination of prostatic cancer in men at the age of fifty or above. The test is also suitable for continuous monitoring of t-PSA-specific antigen, and is helpful for the treatment and management of prostatic cancer patients.

1. Appearance and physical and chemical properties of the preparation

Similar to the preparation in the example 1, the preparation provided in this example is composed of uniform and smooth solid microspheres. When purified water is added to the preparation, the microspheres were disintegrated and re-dissolved quickly and formed a uniform suspension with magnetic bead powder. The magnetic beads didn't agglomerate, and the liquid had no other undissolved substance except the magnetic bead powder.

2. Precision testing

Low-value, mid-value, and high value enterprise references were used, the test was carried out for 8 times in parallel at each concentration, and the mean values x̅ and standard deviations (SD) were calculated respectively, and CV=x̅/SD*100%.

Result: the intra-batch coefficient of variation (CV) was smaller than or equal to 5%. See Table 7 for the intra-batch precision test result. As calculated, the tested CV was 4.4% for the low-value quality control; 1.5% for the mid-value quality control; and 3.1% for the high-value quality control.

TABLE 7
Test Result of Intra-Batch Precision in Example 2

| | | | | | | | | |
|---|---|---|---|---|---|---|---|---|
| Low-value quality control | 25126 | 24185 | 26060 | 27564 | 25617 | 24960 | 27199 | 25888 |
| Mid-value quality control | 112027 | 111944 | 110999 | 112185 | 112605 | 110255 | 115410 | 110706 |
| High-value quality control | 2659771 | 2757514 | 2761693 | 2604084 | 2846512 | 2779399 | 2646242 | 2677880 |

3. Linearity range

Standard samples (100 ng/mL) close to the upper limit of the linearity range were diluted with normal human serum to 6 different concentrations, among which the minimum concentration was 0.02 ng/mL, which was close to the lower limit of the linearity range. Each sample was tested for 2 times, the mean value was calculated, and the relative luminescence intensity was fitted linearly.

Result: as shown in Table 8, the linear coefficient of correlation R was greater than 0.999 in the linearity range (0.02-100 ng/mL) of the freeze-dried microsphere reagent (i.e., the freeze-dried preparation of chemiluminescent immune microspheres), exhibiting close correlation.

TABLE 8
Test Result of Linearity Range and Relative Luminescent
Intensity in Example 2

| Reagent | Concentration, | Freeze-dried microsphere reagent |
|---|---|---|
| No. | ng/mL | Relative luminescent intensity (RLU) |
| 1 | 0 | 260 |
| 2 | 0.02 | 1799 |
| 3 | 1 | 41138 |
| 4 | 3 | 115566 |
| 5 | 10 | 306314 |
| 6 | 30 | 815953 |
| 7 | 100 | 2606467 |
| R | | 0.9994 |

4. Stability of the preparation in storage at 45° C.

The preparation was stored in an incubator at 45° C., and the quality control was tested at different storage times, and compared with the 0-day test result. The quality control was prepared at the same time, and sub-packaged into centrifuge tubes and stored in a frozen state at −20° C., and a fresh tube of quality control was taken out and thawed whenever the quality control was tested.

Result: the preparation had high stability during the storage at a high temperature, and the deviation of the quality control was always within 8%. See Table 9 for the test result.

TABLE 9
Test Result of the Stability of the Preparation Provided in
Example 2 during Storage at 45° C.

| | | Day 90 of storage | |
|---|---|---|---|
| | | | Deviation from the value of day 0 (after |
| Storage time | Day 0 of storage | RLU | the background was removed) |
| Detection background | 244 | 501 | |
| Quality control 1 | 1120 | 1385 | 0.9 |
| Quality control 2 | 45450 | 49191 | 7.7 |
| Quality control 3 | 1138473 | 1226281 | 7.7 |

In summary, when the preparation provided in this example is used to test and analyze the total prostate (t-PSA) specific antigen in serum and/or plasma, the performance analysis result indicates: the intra-batch coefficient of variation (CV) is smaller than or equal to 5%; the linearity range is 0.02-100 ng/mL, and the linear correlation coefficient (r) is greater than or equal to 0.999; and the change ratio of stability of the reagent after storage at 45° C. for 90 days is smaller than or equal to 8%.

### Example 3

Example 3 provides a freeze-dried preparation of chemiluminescent immune microsphere and a test kit for quantitatively measuring the content of thyroid stimulating hormone (TSH) in human serum in vitro.

Thyroid stimulating hormone (TSH) is a glycoprotein secreted by pituitary cells, and includes an a subunit and a 13 subunit, wherein the 13 subunit is a functional subunit. The secretion of TSH is regulated by the TSH releasing hormone secreted by the hypothalamus and the feedback of the thyroid hormone in blood circulation, and is biorhythmic.

The TSH test is a preliminary screening test for evaluating the thyroid function. A subtle change in the concentration of free thyroxine will lead to significant regulation of the TSH concentration in the reversed direction. Therefore, the TSH level is a very sensitive and specific parameter to assist the evaluation of thyroid function, and the TSH test is especially suitable for early detection or exclusion of hypothalamus-pituitary-thyroid axis dysfunction. The TSH test is also used clinically to assist the assessment on the diagnosis and treatment result of primary hyperthyroidism and hypothyroidism, and the TSH level in the patient also vary in the patient with secondary hyperthyroidism or hypothyroidism, depending on the site of the primary disease.

1. Appearance and physical and chemical properties of the preparation

Similar to the preparation in the examples 1 and 2, the preparation provided in this example is composed of uniform and smooth solid microspheres. When purified water is added to the preparation, the microspheres were disintegrated and re-dissolved quickly and formed a uniform suspension with magnetic bead powder. The magnetic beads didn't agglomerate, and the liquid had no other undissolved substance except the magnetic bead powder.

2. Precision testing

Low-value, mid-value, and high value enterprise references were used, the test was carried out for 10 times in parallel at each concentration, and the mean values x̅ and standard deviations (SD) were calculated respectively, and CV=x̅/SD*100%.

Result: the intra-batch coefficient of variation (CV) was smaller than or equal to 6%. See Table 10 for the intra-batch precision test result. As calculated, the tested CV was 5.5% for the low-value quality control; 4.6% for the mid-value quality control; and 3.4% for the high-value quality control.

TABLE 10
Test Result of Intra-Batch Precision in Example 3

| | | | | | | | | | | |
|---|---|---|---|---|---|---|---|---|---|---|
| Low-value | 1716 | 1804 | 1722 | 1903 | 1670 | 1778 | 1735 | 1843 | 1971 | 1901 |
| quality control | | | | | | | | | | |
| Mid-value | 81609 | 82423 | 79423 | 72391 | 82955 | 81894 | 75430 | 79957 | 83744 | 82339 |
| quality control | | | | | | | | | | |
| High-value | 1375719 | 1420326 | 1301410 | 1353346 | 1402974 | 1324529 | 1429669 | 1396950 | 1443193 | 1390767 |
| quality control | | | | | | | | | | |

3. Linearity range

Standard samples (100 ng/mL) close to the upper limit of the linearity range were diluted with normal human serum to 6 different concentrations, among which the minimum concentration was 0.15 ng/mL. Each sample was tested for 2 times, the mean value was calculated, and the relative luminescence intensity was fitted linearly.

Result: as shown in FIG. 11, in the linearity range (0.15-100 ng/mL) of the freeze-dried microspheres, the linearly correlation coefficient R was greater than 0.999, which exhibited close correlation.

TABLE 11
Test Result of Linearity Range and Relative Luminescent
Intensity in Example 3

| Reagent | Concentration, | Freeze-dried microsphere reagent |
|---|---|---|
| No. | ng/mL | Relative luminescent intensity (RLU) |
| 1 | 0 | 205 |
| 2 | 0.15 | 2890 |
| 3 | 1.5 | 27317 |
| 4 | 15 | 265589.5 |
| 5 | 100 | 1904965.5 |
| R | | 0.9999 |

4. Stability of the preparation in storage at 45° C.

The preparation was stored in an incubator at 45° C., and the quality control was tested at different storage times, and compared with the 0-day test result. The quality control was prepared at the same time, and sub-packaged into centrifuge tubes and stored in a frozen state at −20° C., and a fresh tube of quality control was taken out and thawed whenever the quality control was tested.

Result: the preparation had high stability during the storage at a high temperature, and the deviation of the quality control was always within 8%. See Table 12 for the test result.

TABLE 12
Test Result of the Stability of the Preparation Provided
in Example 3 during Storage at 45° C.

| | | Day 86 of storage | |
|---|---|---|---|
| | | | Deviation from the value of day 0 (after |
| Storage time | Day 0 of storage | RLU | the background was removed) |
| Detection background | 139.6 | 228.2 | |
| Quality control 1 | 1812 | 1910.6 | 0.6 |
| Quality control 2 | 79760.2 | 85564 | 7.2 |
| Quality control 3 | 1383888.3 | 1476186 | 6.7 |

In summary, when the preparation provided in this example is used to quantitatively measure the content of thyroid stimulating hormone (TSH) in human serum in vitro, the test result indicates that the intra-batch coefficient of variation (CV) is smaller than or equal to 6%; the linearity range is 0.15-100 ng/mL, and the linear correlation coefficient (r) is greater than or equal to 0.999; and the change ratio of stability of the reagent after storage at 45° C. for 86 days is smaller than or equal to 8%.

### Example 4

Example 4 provides a freeze-dried preparation of chemiluminescent immune microsphere and a test kit for quantitatively measuring the content of cardiac troponin I (CTNI) in human serum in vitro.

cTnI is mainly stored in the myocardial muscle tissues, but doesn't exist in the skeletal muscles or visceral smooth muscles in human body. Under normal conditions, cTnI is hardly detectable in the blood. Troponin is a regulatory protein in the myocardial muscle tissues, and its main function is to participate in the contraction of myocardium. Elevated cTnI means a myocardial injury, which is often caused by the heart itself or other diseases. cTnI is used for auxiliary clinical diagnosis, risk stratification and prognosis evaluation of patients with acute myocardial infarction.

1. Appearance and physical and chemical properties of the preparation

Similar to the preparation in the examples 1-3, the preparation provided in this example is composed of uniform and smooth solid microspheres. When purified water is added to the preparation, the microspheres were disintegrated and re-dissolved quickly and formed a uniform suspension with magnetic bead powder. The magnetic beads didn't agglomerate, and the liquid had no other undissolved substance except the magnetic bead powder.

2. Precision testing

Low-value, mid-value, and high value enterprise references were used, the test was carried out for 8 times in parallel at each concentration, and the mean values \hat{x} and standard deviations (SD) were calculated respectively, and CV=\hat{x}/SD*100%.

Result: the intra-batch coefficient of variation (CV) was smaller than or equal to 6%. See Table 13 for the intra-batch precision test result. As calculated, the tested CV was 3.9% for the low-value quality control; 3.6% for the mid-value quality control; and 5.3% for the high-value quality control.

TABLE 13
Test Result of Intra-Batch Precision in Example 4

| | | | | | | | | | | |
|---|---|---|---|---|---|---|---|---|---|---|
| Low-value | 5127 | 4911 | 5070 | 4860 | 4655 | 5035 | 5190 | 4749 | 5181 | 5173 |
| quality control | | | | | | | | | | |
| Mid-value | 211402 | 230576 | 228395 | 236551 | 215328 | 219196 | 221059 | 213846 | 225576 | 222456 |
| quality control | | | | | | | | | | |
| High-value | 3000248 | 3150949 | 2999042 | 2890001 | 2957357 | 3085279 | 2682852 | 3239993 | 3118219 | 3113297 |
| quality control | | | | | | | | | | |

3. Linearity range

Standard samples (100 ng/mL) close to the upper limit of the linearity range were diluted with normal human serum to 6 different concentrations, among which the minimum concentration was 0.1 ng/mL, which was close to the lower limit of the linearity range. Each sample was tested for 2 times, the mean value was calculated, and the relative luminescence intensity was fitted linearly.

Result: as shown in Table 14, the linear coefficient of correlation R was greater than 0.999 in the linearity range (0.10-100 ng/mL) of the freeze-dried microsphere reagent (i.e., the freeze-dried preparation of chemiluminescent immune microspheres), exhibiting close correlation.

TABLE 14
Test Result of Linearity Range and Relative Luminescent
Intensity in Example 4

| Reagent | Concentration, | Freeze-dried microsphere reagent |
|---|---|---|
| No. | ng/mL | Relative luminescent intensity (RLU) |
| 1 | 0 | 478 |
| 2 | 0.1 | 5537 |
| 3 | 1.2 | 53308.5 |
| 4 | 14 | 493014 |
| 5 | 100 | 3453635 |
| R | | 0.9998 |

4. Stability of the preparation in storage at 45° C.

The preparation was stored in an incubator at 45° C., and the quality control was tested at different storage times, and compared with the 0-day test result. The quality control was prepared at the same time, and sub-packaged into centrifuge tubes and stored in a frozen state at −20° C., and a fresh tube of quality control was taken out and thawed whenever the quality control was tested.

Result: the preparation had high stability during the storage at a high temperature, and the deviation of the quality control was always within 5%. See Table 15 for the test result.

TABLE 15
Test Result of the Stability of the Preparation Provided
in Example 4 during Storage at 45° C.

| | | Day 86 of storage | |
|---|---|---|---|
| | | | Deviation from the value of day 0 (after |
| Storage time | Day 0 of storage | RLU | the background was removed) |
| Background | 1014.5 | 1232.2 | / |
| Quality control 1 | 4587.8 | 4631.8 | −4.9 |
| Quality control 2 | 235956.6 | 236828.6 | 0.3 |
| Quality control 3 | 3004201.2 | 3105105.4 | 3.4 |

In summary, when the preparation provided in this example is used to quantitatively measure the content of cardiac troponin I (CTNI) in human serum in vitro, the test result indicates that the intra-batch coefficient of variation (CV) is smaller than or equal to 7%; the linearity range is 0.13-100 ng/mL, and the linear correlation coefficient (r) is greater than or equal to 0.999; and the change ratio of stability of the reagent after storage at 45° C. for 86 days is smaller than or equal to 5%.

### Example 5

Example 5 provides a freeze-dried preparation of chemiluminescent immune microsphere and a test kit for quantitatively measuring the content of procalcitonin (PCT) in human serum in vitro.

Procalcitonin (PCT) is the precursor of calcitonin. Under normal conditions, most PCT is synthesized and secreted by thyroid C cells, and a small fraction of PCT is produced by other neuroendocrine cells. The PCT concentration in healthy human blood is very low, but it will increase when a systemic bacterial infection occurs, and the degree of increase is positively correlated with the severity of the infection. PCT detection is the best observation indicator for early differential diagnosis of severe bacterial infection/sepsis, and can assist rapid clinical differentiation of bacterial infection from viral infection, judge the severity of bacterial infection, and assist the start/stop of clinical use of antibiotics, etc.

1. Appearance and Physical and Chemical Properties of the Preparation

Similar to the preparation in the examples 1-4, the preparation provided in this example is composed of uniform and smooth solid microspheres. When purified water is added to the preparation, the microspheres were disintegrated and re-dissolved quickly and formed a uniform suspension with magnetic bead powder. The magnetic beads didn't agglomerate, and the liquid had no other undissolved substance except the magnetic bead powder.

2. Precision Testing

Low-value, mid-value, and high value enterprise references were used, the test was carried out for 10 times in parallel at each concentration, and the mean values x̅ and standard deviations (SD) were calculated respectively, and CV=x̅/SD*100%.

Result: the intra-batch coefficient of variation (CV) was smaller than or equal to 7%. See Table 16 for the intra-batch precision test result. As calculated, the tested CV was 6.7% for the low-value quality control; 1.6% for the mid-value quality control; and 2.0% for the high-value quality control.

TABLE 16
Test Result of Intra-Batch Precision in Example 5

| | | | | | | | | | | |
|---|---|---|---|---|---|---|---|---|---|---|
| Low-value quality | 9708 | 9331 | 8786 | 9275 | 10726 | 8901 | 8737 | 8909 | 9359 | 8700 |
| control | | | | | | | | | | |
| Mid-value quality | 678348 | 677059 | 668200 | 674786 | 657789 | 649892 | 669875 | 677440 | 680303 | 681485 |
| control | | | | | | | | | | |
| High-value | 12865474 | 13165513 | 12612679 | 12604284 | 12457846 | 12760793 | 12740510 | 12806635 | 12622764 | 12244357 |
| quality control | | | | | | | | | | |

3. Linearity Range

Standard samples (100 ng/mL) close to the upper limit of the linearity range were diluted with normal human serum to 6 different concentrations, among which the minimum concentration was 0.13 ng/mL. Each sample was tested for 2 times, the mean value was calculated, and the relative luminescence intensity was fitted linearly.

Result: as shown in Table 17, the linear coefficient of correlation R was greater than 0.999 in the linearity range (0.13-100 ng/mL) of the freeze-dried microsphere reagent (i.e., the freeze-dried preparation of chemiluminescent immune microspheres), exhibiting close correlation.

TABLE 17
Test Result of Linearity Range and Relative Luminescent
Intensity in Example 5

| Reagent | Concentration, | Freeze-dried microsphere reagent |
|---|---|---|
| No. | ng/mL | Relative luminescent intensity (RLU) |
| 1 | 0 | 787 |
| 2 | 0.13 | 10032 |
| 3 | 0.95 | 92450 |
| 4 | 10.58 | 1504818 |
| 5 | 33.9 | 4736688 |
| 6 | 100 | 12613128 |
| R | | 0.9992 |

4. Stability of the Preparation in Storage at 45° C.

The preparation was stored in an incubator at 45° C., and the quality control was tested at different storage times, and compared with the 0-day test result. The quality control was prepared at the same time, and sub-packaged into centrifuge tubes and stored in a frozen state at −20° C., and a fresh tube of quality control was taken out and thawed whenever the quality control was tested.

Result: the preparation had high stability during the storage at a high temperature, and the deviation of the quality control was always within 8%. See Table 18 for the test result.

TABLE 18
Test Result of the Stability of the Preparation Provided
in Example 5 during Storage at 45° C.

| | | Day 86 of storage | |
|---|---|---|---|
| | | | Deviation from the value of day 0 (after |
| Storage time | Day 0 of storage | RLU | the background was removed) |
| Detection background | 385 | 814 | / |
| Quality control 1 | 8808 | 9243.2 | 0.1 |
| Quality control 2 | 657861 | 671517.7 | 2.0 |
| Quality control 3 | 12720930.5 | 12688085.5 | −0.3 |

In summary, when the preparation provided in this example is used to quantitatively measure the content of procalcitonin (PCT) in human serum in vitro, the test result indicates that the intra-batch coefficient of variation (CV) is smaller than or equal to 7%; the linearity range is 0.13-100 ng/mL, and the linear correlation coefficient (r) is greater than or equal to 0.999; and the change ratio of stability of the reagent after storage at 45° C. for 86 days is smaller than or equal to 5%.

The freeze-dried preparation of chemiluminescent immune microspheres prepared in the present invention can be packed individually and stored at room temperature, and has the characteristics of high sensitivity, high specificity, wide linearity range, and high stability, etc., and can meet the need of clinical testing.

The above embodiments are only intended to interpret and illustrate the technical scheme of the present invention, but don't constitute any limitation to the present invention. Although the present invention is described above in embodiments, those skilled in the art should understand that various modifications or equivalent replacements can still be made to the embodiments without departing from the spirit and scope of the present invention, and all of such modifications or equivalent replacements shall be deemed as falling in the scope of protection as defined by the claims.

What is claimed is:

1. A freeze-dried preparation of chemiluminescent immune microspheres, wherein
composed of one or more spherical solid particles having the same composition, comprising a reagent storage agent, a magnetic particle coating raw material, and an acridinium ester marking raw material, wherein the magnetic particle coating raw material is an antibody or an antigen coupled with a magnetic particle, and the acridinium ester marking raw material is an antibody or an antigen marked by an acridinium ester.

2. The freeze-dried preparation of chemiluminescent immune microspheres according to claim 1, wherein the reagent storage agent comprises a freeze-drying protectant, PEG20000 and an antioxidant, wherein the freeze-drying protectant comprises mannitol, trehalose, casein, a surfactant, gelatin, a preservative, and a buffer solution TBS.

3. The freeze-dried preparation of chemiluminescent immune microspheres according to claim 2, wherein based on the total mass percentage of the freeze-drying protectant, which is regarded as 100%, the mass percentages of the components in the freeze-drying protectant are as follows: the mannitol: 2%-10%; the trehalose: 5%-20%; the casein: 0.5%-2%; the surfactant: 0.05%-0.5%; the gelatin: 0.05%-1%; the preservative: 0.1%-1%; and the buffer solution TBS: 65.5%-92.3%.

4. The freeze-dried preparation of chemiluminescent immune microspheres according to claim 2, wherein
the buffer solution TBS is made of 10-50 mM Tris-HCl and 0.85% NaCl, so that the pH of the buffer solution TBS is 7.0-7.4; the surfactant is Tween 20, Tween 80 or Triton x100; the antioxidant is a mixture of sodium thiosulfate and EDTA-2Na mixed at 1:1 mix ratio.

5. A method for preparing the freeze-dried preparation of chemiluminescent immune microspheres according to claim 1, comprising the following steps:
S1: obtaining a reagent storage agent, which comprises a freeze-drying protectant, PEG20000 and an antioxidant;
S2: obtaining a magnetic particle coating raw material and an acridinium ester marking raw material, wherein the magnetic particle coating raw material is an antibody or an antigen coupled with a magnetic particle, and the acridinium ester marking raw material is an antibody or an antigen marked by an acridinium ester; mixing the magnetic particle coating raw material with the reagent storage agent obtained in the step S1 to form a reagent of magnetic particle coating raw material, and mixing the acridinium ester marking raw material with the reagent storage agent obtained in the step S1 to form a reagent of acridinium ester marking raw material;
S3: mixing the reagent of magnetic particle coating raw material and the reagent of acridinium ester marking raw material that are obtained in the step S2 at a mix ratio of 1:1 to form a mixed solution, spotting the mixed solution on a liquid nitrogen spotter to form frozen microspheres, and then transferring the frozen microspheres into a freeze drier and freeze-drying the frozen microspheres in vacuum, so as to obtain the freeze-dried preparation of microspheres composed of spherical solid particles.

6. The preparation method according to claim 5, further comprising the following step:
S4: charging a protective gas to the freeze-dried preparation of microspheres composed of spherical solid particles that are obtained in the step S3, sub-packaging the freeze-dried preparation of microspheres into individual packs, and storing the individual packs for assay.

7. An application of the freeze-dried preparation of chemiluminescent immune microspheres according to claim 1 in the preparation of an immunoassay test kit.

8. A chemiluminescent immunoassay test kit, containing the freeze-dried preparation of chemiluminescent immune microspheres according to claim 1.

9. The chemiluminescent immunoassay test kit according to claim 8, further comprising deionized water for disintegrating the freeze-dried preparation of chemiluminescent immune microspheres.

10. An application of the freeze-dried preparation of chemiluminescent immune microspheres according to claim 1, wherein the application is an immunoassay that is not for a disease diagnosis and treatment purpose, and the test item is the content of N-terminal pro-brain natriuretic peptide NT-proBNP, or the content of total prostate specific antigen PSA, or the content of thyroid-stimulating hormone TSH, or the content of cardiac troponin-I cTnI, or the content of procalcitonin PCT in serum or plasma.

11. An application of the chemiluminescent immunoassay test kit according to claim 8, wherein the application is an immunoassay that is not for a disease diagnosis and treatment purpose, and the test item is the content of N-terminal pro-brain natriuretic peptide NT-proBNP, or the content of total prostate specific antigen PSA, or the content of thyroid-stimulating hormone TSH, or the content of cardiac troponin-I cTnI, or the content of procalcitonin PCT in serum or plasma.
